(19) United States
(12) Patent Application Publication
(10) Pub. No.: US 2017/0156659 A1
(43) Pub. Date: Jun. 8, 2017

(54) DEVICE, SHOES, INSOLES AND METHOD FOR CORRECTING WALKING AND STANDING POSTURES

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Ming YANG, Beijing (CN); Xiaochuan CHEN, Beijing (CN); Lei WANG, Beijing (CN); Rui XU, Beijing (CN); Pengcheng LU, Beijing (CN); Qian WANG, Beijing (CN); Peng LIU, Beijing (CN); Yingming LIU, Beijing (CN); Xuebo ZHANG, Beijing (CN); Haisheng WANG, Beijing (CN); Jiantao LIU, Beijing (CN)

(73) Assignees: BOE TECHNOLOGY GROUP CO., LTD., Beijing (CN); BEIJING BOE OPTOELECTRONICS TECHNOLOGY CO., LTD., Beijing (CN)

(21) Appl. No.: 15/022,717

(22) PCT Filed: Oct. 22, 2015
(86) PCT No.: PCT/CN2015/092492

§ 371 Date: Mar. 17, 2016

(30) Foreign Application Priority Data
Jun. 25, 2015 (CN) 201510357718.0

(51) Int. Cl. A61B 5/00; G09B 19/00; A61B 5/11; A43B 17/00; A43B 3/00

(52) U.S. Cl. CPC A61B 5/486; A43B 17/00; A43B 3/0021; A43B 3/001; A61B 5/112; A61B 5/6807; G09B 19/003

(57) ABSTRACT

Embodiments of the present disclosure provide a device, shoes, insoles and a method for correcting walking and standing postures. The device for correcting walking postures includes: a signal transmitter, to transmit a signal; a signal receiver, wirelessly connected to the signal transmitter, to receive the signal transmitted by the signal transmitter; a processor, connected to the signal receiver, to determine whether strength of the signal received by the signal receiver is in a preset strength range and generate a prompting signal if the strength of the signal received by the signal receiver is not in the preset strength range; and a prompter, connected to the processor, to prompt a user that the walking posture is not right after receiving the prompting signal.

### CROSS REFERENCE OF RELATED APPLICATION

This application claims a priority to Chinese Patent Application No. 201510357718.0 filed on Jun. 25, 2015, the disclosures of which are incorporated in their entirety by reference herein.

### TECHNICAL FIELD

The present disclosure relates to the field of health care, and more particularly, to a device, shoes, insoles and a method for correcting walking and standing postures.

### BACKGROUND

Right walking and standing postures are not only beautiful, but may ensure human health. Wrong walking and standing postures, such as in-toe posture and out-toe posture are not only not beautiful, but also may severely wear socks and shoes and may affect human health in the long run.

At present, there are some health shoes, which may assist in correcting users' walking and standing postures after internal structure of the shoes are altered. However, theses health shoes cannot be used to detect whether the waking and standing postures of the user are right and cannot give any prompt.

### SUMMARY

In view of this, embodiments of the present disclosure provide a device, shoes, insoles and a method for correcting walking and standing postures, which may detect whether the walking posture and standing posture of the user are right and give prompts.

In order to solve the above problem, one embodiment of the present disclosure provides a device for correcting a walking posture, including: a signal transmitter, to transmit a signal; a signal receiver, wirelessly connected to the signal transmitter, to receive the signal transmitted by the signal transmitter; a processor, connected to the signal receiver, to determine whether strength of the signal received by the signal receiver is in a preset strength range and generate a prompting signal if the strength of the signal received by the signal receiver is not in the preset strength range; and a prompter, connected to the processor, to prompt a user that the walking posture is not right after receiving the prompting signal.

Optionally, the device for correcting the walking posture further includes: a storage, connected to the processor, to store preset strength ranges corresponding to different walking modes, wherein the walking modes include: one or more of normal walking, fast walking, fast running and jogging; and a mode selector, connected to the storage and the processor, to select one of the walking modes; wherein the processor is further to obtain a preset strength range corresponding to the walking mode currently selected by the mode selector and take the obtained preset strength range as a preset strength range adopted by the processor.

Optionally, the device for correcting the walking posture further includes: a setter, connected to the storage, to set the preset strength ranges, wherein the preset strength ranges are strength ranges of signals received by the signal receiver when walking postures of different walking modes of the user who respectively wears the signal transmitter and the signal receiver on his/her feet are right.

Optionally, the processor is further to, after the strength of the signal received by the signal receiver is determined to exceed the preset strength range, determine whether the walking posture of the user is an out-toe posture or an in-toe posture according to whether the strength of the signal received by the signal receiver is less than the minimum value of the preset strength range or larger than the maximum value of the preset strength range, generate a prompting signal including out-toe information or in-toe information.

Optionally, the processor is further to, after the strength of the signal received by the signal receiver is determined to exceed the preset strength range, determine a deviation level of the walking posture of the user according to a deviation value between the strength of the signal received by the signal receiver and the preset strength range and generate a prompting signal including the deviation level.

Optionally, the signal transmitter is a Radio Frequency (RF) signal transmitter and the signal receiver is an RF signal receiver.

Optionally, the signal receiver, the processor and the prompter are integrated in a same circuit chip.

Optionally, the device for correcting the walking posture further includes: an electronic device, in which the processor and the prompter are set.

Optionally, the prompter includes one or more of a display, a vibrator, a sound player or a prompting light.

Optionally, the electronic device is a wearable electronic device or a handheld electronic device.

Optionally, the signal transmitter and the signal receiver are simultaneously set at forefeet or simultaneously set at heels.

One embodiment of the present disclosure provides a device for correcting a standing posture, including: a signal transmitter, to transmit a signal; a signal receiver, wirelessly connected to the signal transmitter, to receive the signal transmitted by the signal transmitter; a processor, connected to the signal receiver, to determine whether strength of the signal received by the signal receiver is in a preset strength range and generate a prompting signal if the strength of the signal received by the signal receiver is not in the preset strength range; and a prompter, connected to the processor, to prompt a user that the standing posture is not right after receiving the prompting signal.

One embodiment of the present disclosure further provides a pair of shoes, including the above device for correcting the standing/walking posture.

One embodiment of the present disclosure further provides a pair of insoles including the above device for correcting the standing/walking posture.

One embodiment of the present disclosure provides a method for correcting a walking posture, adopting the above device for correcting the walking posture and including: transmitting a signal using a signal transmitter, which is worn on a foot of a user, and receiving the signal transmitted by the signal transmitter using a signal receiver, which is worn on another foot of the user; determining whether strength of the signal received by the signal receiver is in a preset strength range and generating a prompting signal if the strength of the signal received by the signal receiver is not in the preset strength range; and prompting the user that the walking posture is not right according to the prompting signal.

Optionally, before determining whether the strength of the signal received by the signal receiver is in the preset strength range, the method further includes: storing preset strength ranges corresponding to different walking modes, wherein the walking modes include: one or more of normal walking, fast walking, fast running and jogging; selecting one of the walking modes; wherein determining whether the strength of the signal received by the signal receiver is in the preset strength range includes: obtaining a preset strength range corresponding to the currently-selected walking mode and determining whether the strength of the signal received by the signal receiver is in the preset strength range corresponding to the currently-selected walking mode.

Optionally, before storing the preset strength ranges corresponding to the different walking modes, the method further includes: setting the preset strength ranges, wherein the preset strength ranges are strength ranges of signals received by the signal receiver when walking postures of different walking modes of the user who respectively wears the signal transmitter and the signal receiver on his/her feet are right.

Optionally, when determining that the strength of the signal received by the signal receiver exceeds the preset strength range, generating the prompting signal specifically includes: after the strength of the signal received by the signal receiver is determined to exceed the preset strength range, determining whether the walking posture of the user is an out-toe posture or an in-toe posture according to whether the strength of the signal received by the signal receiver is less than the minimum value of the preset strength range or larger than the maximum value of the preset strength range and generating a prompting signal including out-toe information or in-toe information.

Optionally, when determining that the strength of the signal received by the signal receiver exceeds the preset strength range, a step for generating the prompting signal specifically includes: after the strength of the signal received by the signal receiver is determined to exceed the preset strength range, determining a deviation level of the walking posture of the user according to a deviation value between the strength of the signal received by the signal receiver and the preset strength range and generating the prompting signal including the deviation level.

One embodiment of the present disclosure provides a method for correcting a standing posture, adopting the above device for correcting the standing posture and including: transmitting a signal using a signal transmitter, which is worn on a foot of a user, and receiving the signal transmitted by the signal transmitter using a signal receiver, which is worn on another foot of the user; determining whether strength of the signal received by the signal receiver equals to a preset strength range and generating a prompting signal if the strength of the signal received by the signal receiver does not equal to the preset strength range; and prompting the user that the standing posture is not right according to the prompting signal.

Advantages of the above technical scheme provided by embodiments of the present disclosure are as follows: determining whether the distance between the feet of the user is the distance between the feet when the walking posture or standing posture is right, that is, determining whether the walking posture or standing posture of the user is right by detecting the strength of the signal received by the signal receiver when the user is walking or standing using the signal transmitter and signal receiver, which are respectively worn on the feet of the user and prompting the user when detecting that the walking posture or standing posture of the user is not right, so that the user may timely correct the walking posture or the standing posture.

### BRIEF DESCRIPTION OF THE DRAWINGS

FIG. 1 is a diagram illustrating structure of a device for correcting walking postures in accordance with a first embodiment of the present disclosure;

FIGS. 2 and 3 are diagrams illustrating positions where a signal transmitter and a signal receiver are set in accordance with embodiments of the present disclosure;

FIG. 4 is a diagram illustrating a range of a distance between the signal transmitter and the signal receiver when the walking posture is right;

FIG. 5 is a diagram illustrating a range of a distance between the signal transmitter and the signal receiver when the walking posture is an in-toe posture;

FIG. 6 is a diagram illustrating a range of a distance between the signal transmitter and the signal receiver when the walking posture is an out-toe posture;

FIG. 7 is a diagram illustrating structure of a device for correcting walking postures in accordance with a second embodiment of the present disclosure;

FIG. 8 is a diagram illustrating structure of a device for correcting walking postures in accordance with a third embodiment of the present disclosure;

FIG. 9 is a flow chart illustrating a method for correcting walking postures in accordance with embodiments of the present disclosure;

FIG. 10 is a diagram illustrating a device for correcting walking postures in accordance with a fourth embodiment of the present disclosure;

FIG. 11 is a diagram illustrating a device for correcting standing postures in accordance with one embodiment of the present disclosure;

FIG. 12 is a diagram illustrating a distance between a signal transmitter and a signal receiver when the standing posture is right;

FIG. 13 is a diagram illustrating a distance between the signal transmitter and the signal receiver when the standing posture is an in-toe posture;

FIG. 14 is a diagram illustrating a distance between the signal transmitter and the signal receiver when the standing posture is an out-toe posture; and

FIG. 15 is a flow chart illustrating a method for correcting standing postures in accordance with one embodiment of the present disclosure.

### DETAILED DESCRIPTION

Embodiments of the present disclosure will be described in detail hereinafter with reference to accompanying drawings and embodiments. The following embodiments are used to describe the present disclosure, but not used to limit the scope of the present disclosure.

Referring to FIG. 1, FIG. 1 is a diagram illustrating structure of a device for correcting walking postures in accordance with a first embodiment of the present disclosure. The device for correcting walking postures may include: a signal transmitter 11, configured to transmit a signal; a signal receiver 12, wirelessly connected to the signal transmitter 11 and configured to receive the signal transmitted by the signal transmitter 11; a processor 13, connected to the signal receiver 12 and configured to determine whether strength of the signal received by the signal receiver 12 is in a preset strength range and generate a prompting signal if the strength of the signal received by the signal receiver 12 is not in the preset strength range; and a prompter 14, connected to the processor 13 and configured to prompt a user that the walking posture is not right after the prompting signal is received.

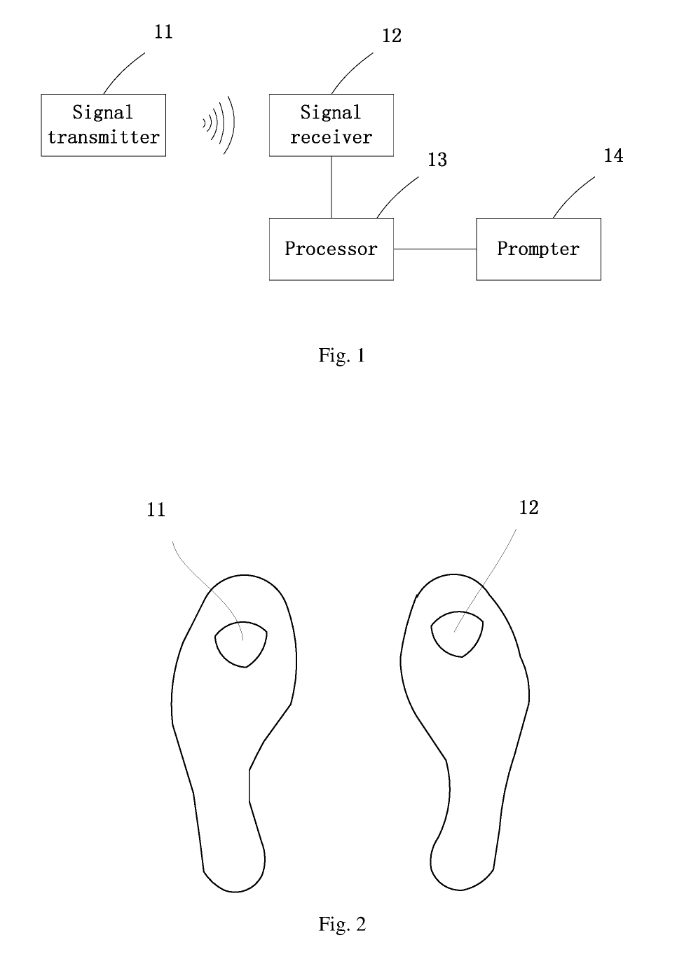

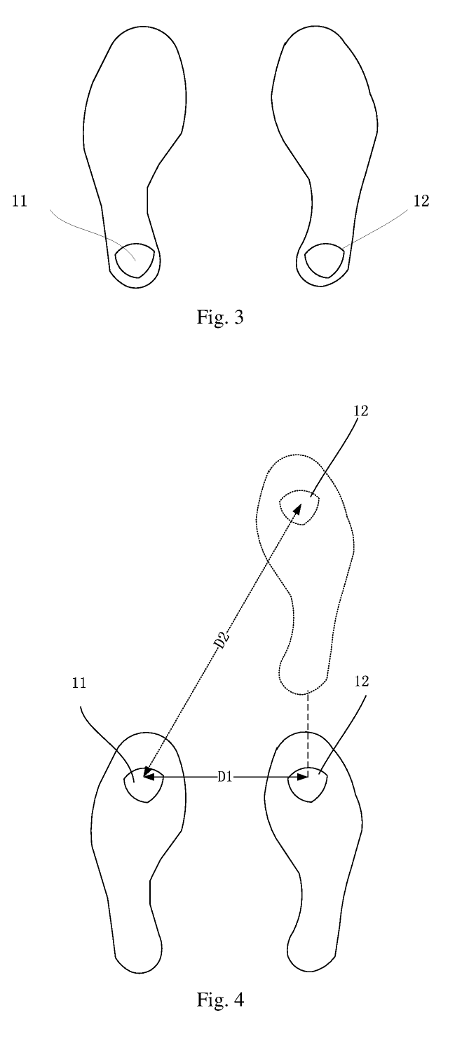

The signal transmitter 11 and signal receiver 12 may be respectively worn on two shoes, insoles and socks of the user or may be directly worn on feet of the user. Optionally, referring to FIG. 2 and FIG. 3, the signal transmitter 11 and the signal receiver 12 are simultaneously set at forefeet or simultaneously set at heels of the user and the signal transmitter 11 and the signal receiver 12 are roughly symmetrically set.

Optionally, the signal transmitted by the signal transmitter 11 may be a Radio Frequency (RF) signal. That is, the signal transmitter 11 may be an RF signal transmitter and the signal receiver 12 may be an RF signal receiver. Certainly, the signal transmitted by the signal transmitter 11 may be other types of signal, such as an infrared signal, etc.

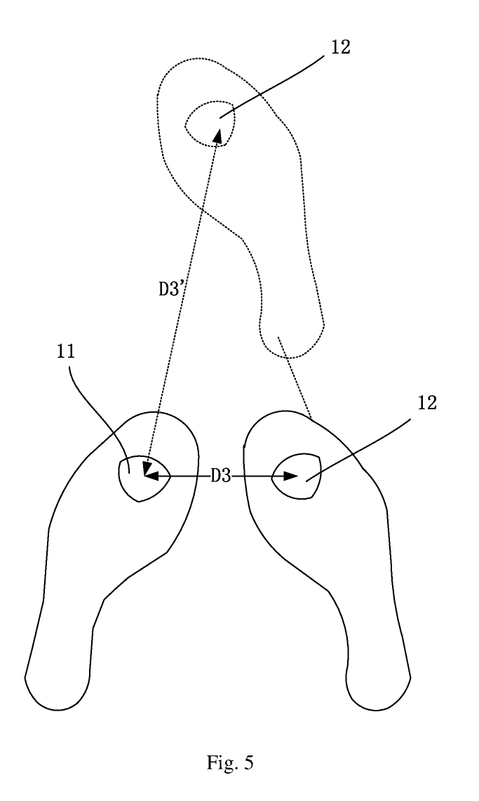

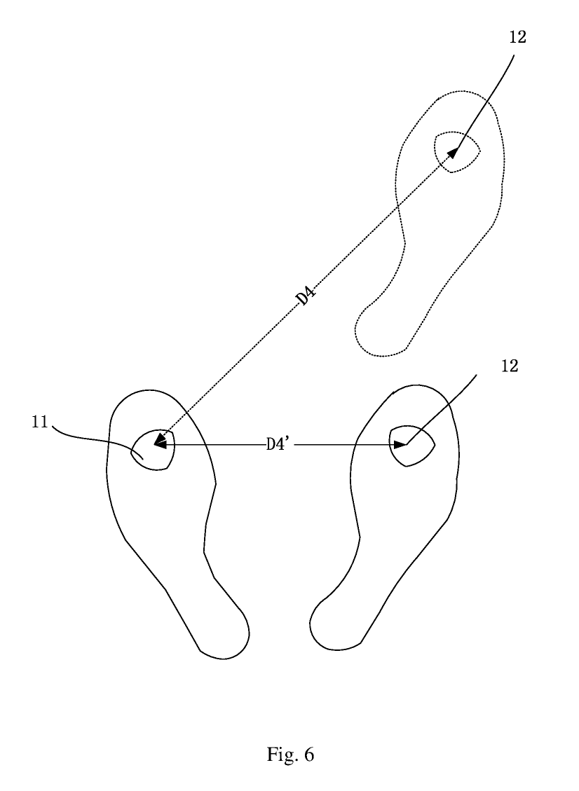

Referring to FIG. 4, when the walking posture of the user is right, in the walking process, feet of the user are approximately parallel. When the feet of the user are closest to each other, the distance between the signal transmitter 11 and the signal receiver 12 is D1. When the feet of the user are farthest from each other, the distance between the signal transmitter 11 and the signal receiver 12 is D2. Referring to FIG. 5, when the walking posture of the user is the in-toe posture, the distance D3 between the signal transmitter 11 and the signal receiver 12 when the feet of the user are closest to each other is less than D1. When the feet of the user are farthest from each other, the distance between the signal transmitter 11 and the signal receiver 12 is D3′. Referring to FIG. 6, when the walking posture of the user is the out-toe posture, the distance between the signal transmitter 11 and the signal receiver 12 is D4′ when the feet of the user are closest to each other and the distance D4 between the signal transmitter 11 and the signal receiver 12 when the feet of the user are farthest from each other is larger than D2.

However, the distance between the signal transmitter 11 and the signal receiver 12 determines the strength of the signal received by the signal receiver 12. The smaller the distance between the signal transmitter 11 and the signal receiver 12, the larger the strength of the signal received by the signal receiver 12. The larger the distance between the signal transmitter 11 and the signal receiver 12, the smaller the strength of the signal received by the signal receiver 12.

Therefore, when the walking posture of the user is right, the strength S1 of the signal, which is received by the signal receiver 12 when the feet of the user are closest to each other, and the strength S2 of the signal, which is received by the signal receiver 12 when the feet of the user are farthest from each other, can be obtained and used to define a preset strength range (S2-S1, where S2<S1). When the walking posture of the user is the in-toe posture, the distance D3 between the signal transmitter 11 and the signal receiver 12 when the feet of the user are closest to each other is less than D1. Therefore, the strength S3 of the signal, which is received by the signal receiver 12 when the in-toed feet of the user are closest to each other, is necessarily larger than S1. However, when the walking posture of the user is the out-toe posture, the distance D4 between the signal transmitter 11 and the signal receiver 12 when the feet of the user are farthest from each other is larger than D2. Therefore, the strength S4 of the signal, which is received by the signal receiver 12 when the out-toed feet of the user are farthest from each other, is necessarily less than S2. In other words, whether the walking posture of the user is right may be determined by comparing the strength of the signal received by the signal receiver 12 with the preset strength range.

One mode for the processor 13 to determine whether the strength of the signal received by the signal receiver 12 is in the preset strength range may be implemented by adopting software or a hardware circuit. When the above mode is implemented by using the hardware circuit, the processor 13 may include a comparing circuit, which is configured to compare an obtained strength of the signal received by the signal receiver 12 with the minimum value and/or maximum value of the preset strength range, generate a prompting signal, such as 1 when the strength of the signal received by the signal receiver 12 is less than the minimum value of the preset strength range or larger than the maximum value of the preset strength range, and generate a normal indicating signal, such as 0 when the strength of the signal received by the signal receiver 12 is larger than the minimum value of the preset strength range, less than the maximum value of the preset strength range, or equals to the minimum value or maximum value of the preset strength range.

The prompter 14 may prompt the user that the walking posture is not right in multiple modes, such as playing sound via a sound player, lighting an indicator light, vibrating using a vibrator and/or displaying on a screen of a display.

The device for correcting walking postures in embodiments of the present disclosure may determine whether the distance between the feet of the user is in the range of the distance between the feet of the right walking posture, i.e., determine whether the walking posture of the user is right by detecting the strength of the signal received by the signal receiver when the user is walking with the signal transmitter and the signal receiver which are respectively worn on the feet of the user. When it is detected that the walking posture of the user is not right, the user is prompted so that the user may correct the walking posture timely.

The daily walking of the user generally includes situations, i.e., walking and running. The ranges of the distances between the feet of the user in different situations are certainly different. Therefore, in embodiments of the present disclosure, different preset strength ranges may be set for different walking modes.

Referring to FIG. 10, the device for correcting walking postures may further include: a storage 15, connected to the processor 13 and configured to store different preset strength ranges corresponding to different walking modes, wherein the walking modes include: one or more of normal walking, fast walking, fast running and jogging; and a mode selector 16, connected to the storage 15 and the processor 13 and configured to select a walking mode.

The processor 13 is further configured to obtain the preset strength range corresponding to the walking mode currently selected by the mode selector 16 and take the obtained preset strength range as the preset strength range adopted by the processor 13.

Optionally, the device for correcting walking postures may further include: a setter 17, connected to the storage 15 and configured to set the preset strength ranges. The preset strength ranges are the strength ranges of signals received by the signal receiver when walking postures of different walking modes of the user who respectively wears the signal transmitter 11 and the signal receiver 12 on his/her feet are right.

Further, it can be understood that as for users of different sizes, the ranges of the distances between the feet are different. As for different users, the above preset strength ranges are also different. Therefore, optionally, the storage 15 may be further configured to store the preset strength ranges corresponding to different users and the preset strength ranges corresponding to the different users may include the preset strength ranges corresponding to different walking modes.

Referring to FIG. 5, when the walking posture of the user is the in-toe posture and the feet of the user are closest to each other, the distance D3 between the signal transmitter 11 and the signal receiver 12 is less than the distance D1 when the walking posture is the right walking posture and the feet of the user are closest to each other. At this point, the strength of the signal received by the signal receiver 12 is larger than the maximum value of the preset strength range. Referring to FIG. 6, when the walking posture of the user is the out-toe posture and the feet of the user are farthest from each other, the distance D4 between the signal transmitter 11 and the signal receiver 12 is larger than the distance D2 when the walking posture is the right walking posture and the feet of the user are farthest from each other. At this point, the strength of the signal received by the signal receiver 12 is less than the minimum value of the preset strength range.

Based on the above analysis, after the strength of the signal received by the signal receiver 12 is determined to exceed the preset strength range, the processor 13 in embodiments of the present disclosure may determine whether the walking posture of the user is the out-toe posture or the in-toe posture according to whether the strength of the signal received by the signal receiver 12 is less than the minimum value of the preset strength range or larger than the maximum value of the preset strength range, generate a prompting signal including out-toe information or in-toe information. The prompting operation is performed by the prompter 14.

For instance, when the prompter 14 is a sound player, and receives a prompting signal including the in-toe, the prompter 14 may play a prompting tone “in-toe”, a beeping sound representing the in-toe or other prompting sound. When the prompter 14 is a display and receives a prompting signaling including the in-toe, the prompter 14 may display the “in-toe” on the display or display other display information. When the prompter 14 is an indicator light, the prompter 14 may include the indicator lights of different colors (such as, a red light represents the in-toe, a green light represents the out-toe). When the prompting signaling including the in-toe is received, the red light is lit up.

The user may realize more clearly that there is something wrong with his/her walking posture via the above prompting signal including the in-toe information or the out-toe information.

Further, when the strength of the signal received by the signal receiver 12 is determined to exceed the preset strength range, the processor 13 may determine a deviation level of the walking posture of the user according to a deviation value between the strength of the signal received by the signal receiver 12 and the preset strength range and generate a prompting signal including the deviation level. The prompting operation is performed by the prompter 14.

For instance, the deviation level may be indicated using numbers. For instance, the in-toe walking posture may be divided into “level 1”, “level 2” and “level 3”. The out-toe walking posture may be divided into “level −1”, “level −2” and level “−3”. The larger the absolute value, the more severe the deviation. Certainly, the deviation level may be indicated using other modes, such as “severe”, “slight”, etc.

When the user corrects the walking posture, the above prompting signal including the deviation level may give a reference to improve the user's experience.

The signal transmitter 11 and signal receiver 12 in the above embodiments may be set on the shoes, insoles or socks of the user in a detachable way, such as may be fixed on a vamp of the shoes using a pin or Velcro, etc. Further, the signal transmitter 11 and the signal receiver 12 also may be set in the shoes or insoles of the user.

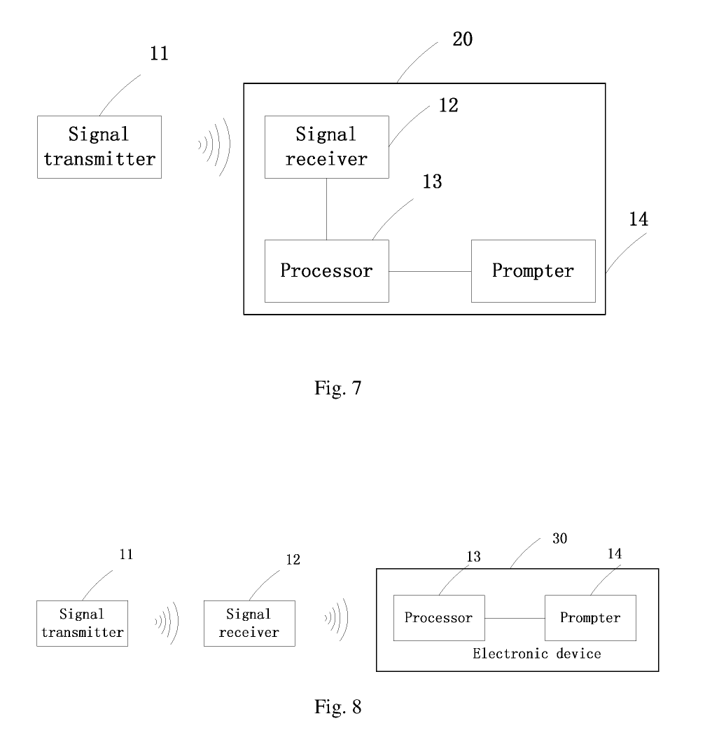

Please referring to FIG. 7, in one embodiment of the present disclosure, the above processor 13, the prompter 14 and the signal receiver 12 may be integrated in a same circuit chip 20. In this situation, the processor 13 connects to the signal receiver 12 in a wired mode. The circuit chip 20 may be set in the shoes or insoles of the user. In this embodiment, the prompter 14 may be the sound player and may prompt the user via playing sound.

Referring to FIG. 8, in another embodiment of the present disclosure, the device for correcting walking postures may further include: an electronic device 30. The processor 13 and the prompter 14 are set in the electronic device 30. That is, the processor 13 and the prompter 14 are separate from the signal receiver 12. The processor 13 connects to the signal receiver 12 in a wireless mode, such as connects to the signal receiver 12 using the wireless mode, such as an RF mode or a Bluetooth mode. The electronic device 30 may be a wearable electronic device, such as a smart watch, a smart band and an electronic hanging, etc. The electronic device 30 may also be a handheld electronic device, such as a cell phone, etc.

The electronic device 30 may provide much more prompting modes. For instance, the prompter 14 may include one or more of a display, a vibrator, a sound player or a prompting light. When the prompter 14 is the vibrator, the device for correcting walking postures may be used by blind or deaf users.

Embodiments of the present disclosure may further provide a kind of shoes, which include the signal transmitter and signal receiver in any of the above embodiments.

When the processor, the prompter and the signal receiver are integrated in a same circuit chip, both the processor and the prompter are set on the shoes.

When the processor and the prompter are set in an electronic device, the shoes need to cooperate with the electronic device to implement the correction of the walking postures.

Embodiments of the present disclosure may further provide a kind of insoles, which include the signal transmitter and signal receiver in any of the above embodiments.

Similarly, when the processor, the prompter and the signal receiver are integrated in a same circuit chip, both the processor and the prompter are set on the insoles.

When the processor and the prompter are set in an electronic device, the shoes need to cooperate with the electronic device to implement the correction of the walking postures.

Optionally, the signal transmitter and the signal receiver are simultaneously set at forefeet of the insoles or simultaneously set at heels of the insoles.

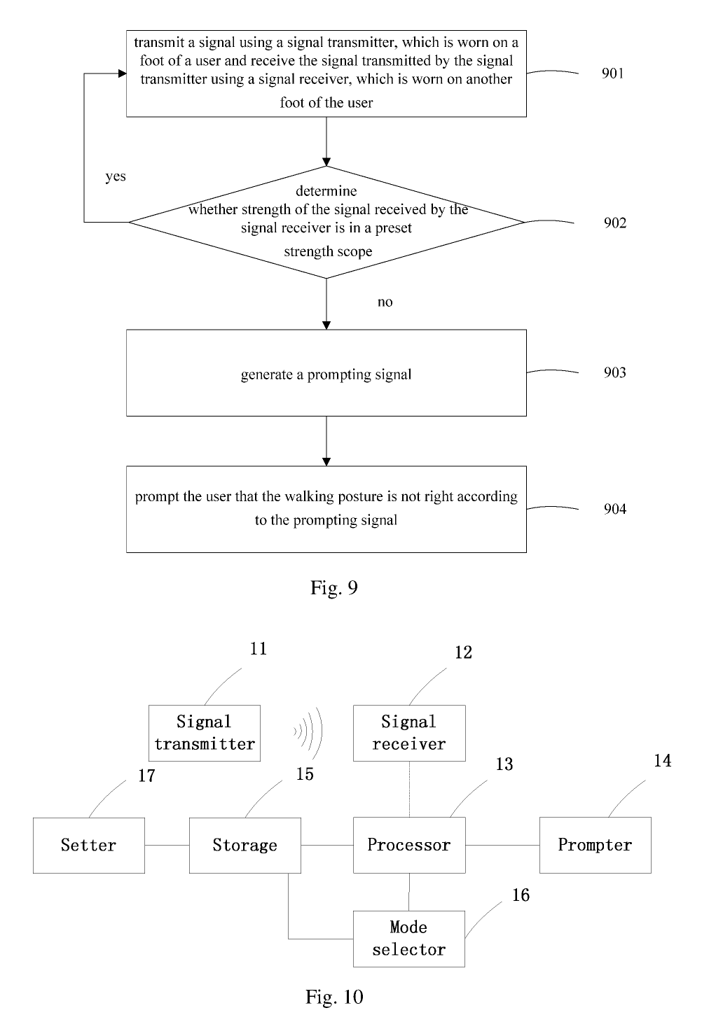

Referring to FIG. 9, embodiments of the present disclosure further provide a method for correcting walking postures, which is applied to the device for correcting walking postures in any of the above embodiments. The method may include: block 901: transmitting a signal using a signal transmitter, which is worn on a foot of a user and receiving the signal transmitted by the signal transmitter using a signal receiver, which is worn on another foot of the user; block 902: determining whether strength of the signal received by the signal receiver is in a preset strength range, executing block 903 if not and continuing to execute the block 901 if yes; block 903: generating a prompting signal; and, block 904: prompting the user that the walking posture is not right according to the prompting signal.

The daily walking of the user generally includes two situations, i.e., walking and running. The ranges of the distances between the feet of the user in different situations are certainly different. Therefore, in embodiments of the present disclosure, different preset strength ranges may be set for different walking modes.

That is, optionally, before determining whether the strength of the signal received by the signal receiver is in the preset strength range, the method further includes: storing preset strength ranges corresponding to different walking modes, wherein the walking modes include: one or more of normal walking, fast walking, fast running and jogging; selecting a walking mode.

Determining whether the strength of the signal received by the signal receiver is in the preset strength range includes: obtaining a preset strength range corresponding to the currently-selected walking mode and determining whether the strength of the signal received by the signal receiver is in the preset strength range corresponding to the currently-selected walking mode.

Optionally, before storing the preset strength ranges corresponding to the different walking modes, the method further includes: setting the preset strength ranges, wherein the preset strength ranges are strength ranges of signals received by the signal receiver when walking postures of different walking modes of the user who respectively wears the signal transmitter and the signal receiver on his/her feet are right.

In order to improve the user's experiences, optionally, when determining that the strength of the signal received by the signal receiver exceeds the preset strength range, the step for generating the prompting signal specifically includes: after the strength of the signal received by the signal receiver is determined to exceed the preset strength range, determining whether the walking posture of the user is an out-toe posture or an in-toe posture according to whether the strength of the signal received by the signal receiver is less than the minimum value of the preset strength range or larger than the maximum value of the preset strength range and generating a prompting signal including out-toe information or in-toe information.

In order to improve the user's experiences, optionally, when determining that the strength of the signal received by the signal receiver exceeds the preset strength range, the step for generating the prompting signal specifically includes: after the strength of the signal received by the signal receiver is determined to exceed the preset strength range, determining a deviation level of the walking posture of the user according to a deviation value between the strength of the signal received by the signal receiver and the preset strength range and generating the prompting signal including the deviation level.

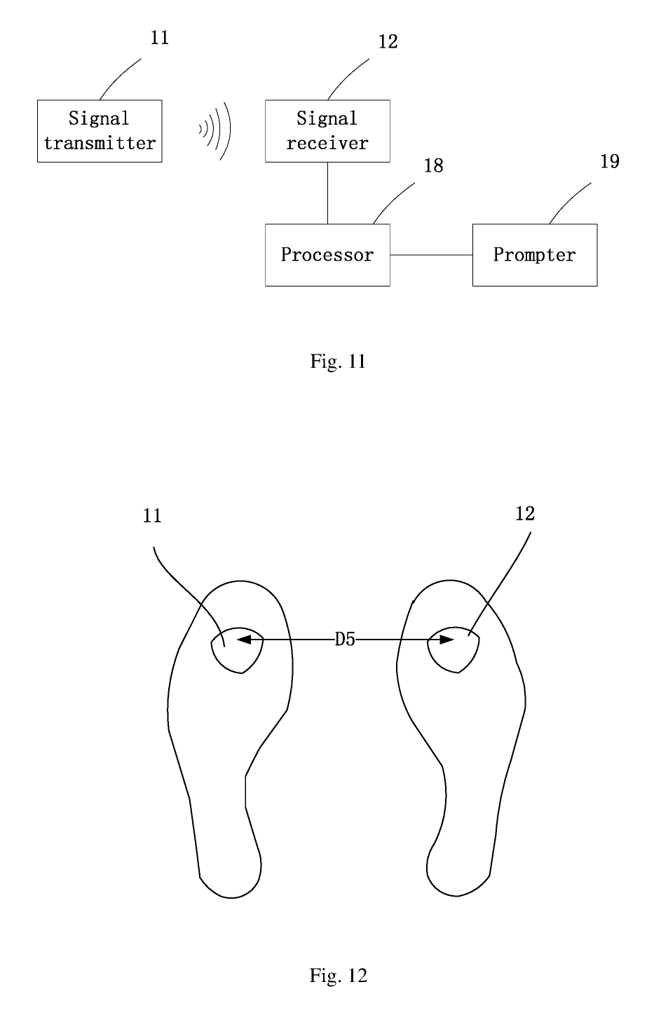

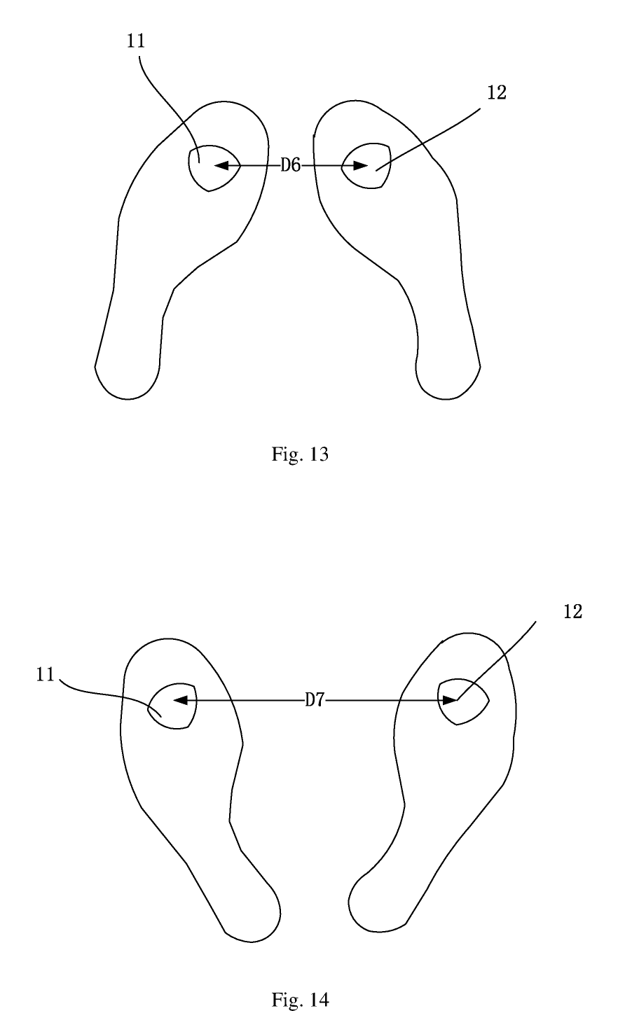

Referring to FIG. 12 to FIG. 14, when a standing posture of the user is right, feet of the user are approximately parallel. The distance between the signal transmitter 11 and the signal receiver 12 is D5. When the standing posture of the user is the in-toe posture, the distance between the signal transmitter 11 and the signal receiver 12 is D6, which is less than D5. When the standing posture of the user is the out-toe posture, the distance between the signal transmitter 11 and the signal receiver 12 is D7, which is larger than D5.

Therefore, when the standing posture of the user is right, the strength of the signal, which is received by the signal receiver 12 is S5. When the standing posture of the user is the in-toe posture, the distance D6 between the signal transmitter 11 and the signal receiver 12 is less than D5. At this moment, the strength S6 of the signal received by the signal receiver 12 is larger than S5. However, when the standing posture of the user is the out-toe posture, the distance D7 between the signal transmitter 11 and the signal receiver 12 is larger than D5. At this moment, the strength S7 of the signal received by the signal receiver 12 is less than S5. In other words, whether the standing posture of the user is right may be determined by comparing the strength of the signal received by the signal receiver 12 with the preset strength.

Based on the above analysis, referring to FIG. 11, one embodiment of the present disclosure further provides a device for correcting standing postures, including: a signal transmitter 11, to transmit a signal; a signal receiver 12, wirelessly connected to the signal transmitter 11, to receive the signal transmitted by the signal transmitter 11; a processor 18, connected to the signal receiver 12, to determine whether strength of the signal received by the signal receiver 12 is in a preset strength range and generate a prompting signal if the strength of the signal received by the signal receiver 12 is not in the preset strength range; and, a prompter 19, connected to the processor 18, to prompt a user that the standing posture is not right after receiving the prompting signal.

One embodiment of the present disclosure further provides a kind of shoes, including the signal transmitter and signal receiver in the embodiment shown in FIG. 11.

The processor, the prompter and the signal receiver may be integrated in a same circuit chip. At this moment, both the processor and the prompter are set in the shoes.

The processor and the prompter may also be set in an electronic device. Then, the shoes need to cooperate with the electronic device to finish the correction of the walking postures.

One embodiment of the present disclosure further provides a kind of insoles, including the signal transmitter and signal receiver in the embodiment shown in FIG. 11.

The processor, the prompter and the signal receiver may be integrated in a same circuit chip. At this moment, both the processor and the prompter are set on the insoles.

The processor and the prompter may also be set in an electronic device. Then, the insoles need to cooperate with the electronic device to finish the correction of the standing postures.

Optionally, the signal transmitter and the signal receiver are simultaneously set at forefeet or simultaneously set at heels.

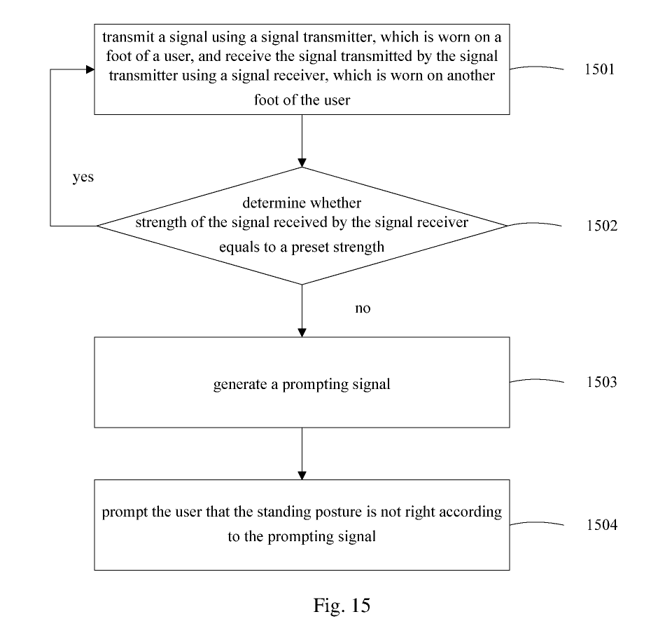

Referring to FIG. 15, one embodiment of the present disclosure further provides a method for correcting standing postures, applied to a device for correcting standing postures, the method includes: block 1501: transmitting a signal using a signal transmitter, which is worn on a foot of a user, and receiving the signal transmitted by the signal transmitter using a signal receiver, which is worn on another foot of the user; block 1502: determining whether strength of the signal received by the signal receiver equals to a preset strength range, executing block 1503 if no and continuing to execute the block 1501 if yes; block 1503: generating a prompting signal; and, block 1504: prompting the user that the standing posture is not right according to the prompting signal.

In the above embodiments, whether the distance between the feet of the user is the distance between the feet when the standing posture is right may be determined, that is, whether the standing posture of the user is right may be determined by detecting the strength of the signal received by the signal receiver when the user is standing using the signal transmitter and signal receiver, which are respectively worn on the feet of the user. The user is prompted when detecting that the standing posture of the user is not right, so that the user may timely correcting the standing posture.

The foregoing description may only describe preferred embodiments of the present disclosure. It should be noted that as for an ordinary skilled in the art of the present disclosure, any modification, or improvement made without departing from the spirit and principle of the present disclosure should be covered by the protection scope of the present disclosure.

1. A device for correcting a walking posture, comprising:
a signal transmitter, to transmit a signal;
a signal receiver, wirelessly connected to the signal transmitter, to receive the signal transmitted by the signal transmitter;
a processor, connected to the signal receiver, to determine whether strength of the signal received by the signal receiver is in a preset strength range and generate a prompting signal if the strength of the signal received by the signal receiver is not in the preset strength range; and
a prompter, connected to the processor, to prompt a user that the walking posture is not right after receiving the prompting signal.

2. The device according to claim 1, further comprising:
a storage, connected to the processor, to store preset strength ranges corresponding to different walking modes, wherein the walking modes comprise: one or more of normal walking, fast walking, fast running and jogging; and
a mode selector, connected to the storage and the processor, to select one of the walking modes;
wherein the processor is further to obtain a preset strength range corresponding to the walking mode currently selected by the mode selector and take the obtained preset strength range as a preset strength range adopted by the processor.

3. The device according to claim 2, further comprising:
a setter, connected to the storage, to set the preset strength ranges, wherein the preset strength ranges are strength ranges of signals received by the signal receiver when walking postures of different walking modes of the user who respectively wears the signal transmitter and the signal receiver on his/her feet are right.

4. The device according to claim 1, wherein the processor is further to, after the strength of the signal received by the signal receiver is determined to exceed the preset strength range, determine whether the walking posture of the user is an out-toe posture or an in-toe posture according to whether the strength of the signal received by the signal receiver is less than the minimum value of the preset strength range or larger than the maximum value of the preset strength range, and generate a prompting signal comprising out-toe information or in-toe information; or,
wherein the processor is further to, after the strength of the signal received by the signal receiver is determined to exceed the preset strength range, determine a deviation level of the walking posture of the user according to a deviation value between the strength of the signal received by the signal receiver and the preset strength range, and generate a prompting signal comprising the deviation level.

5. (canceled)

6. The device according to claim 1, wherein the signal transmitter is a Radio Frequency (RF) signal transmitter and the signal receiver is an RF signal receiver.

7. The device according to claim 1, wherein the signal receiver, the processor and the prompter are integrated in a same circuit chip.

8. The device according to claim 1, further comprising:
an electronic device, in which the processor and the prompter are set wherein the electronic device is a wearable electronic device or a handheld electronic device.

9. The device according to claim 8, wherein the prompter comprises one or more of a display, a vibrator, a sound player or a prompting light.

10. (canceled)

11. The device according to claim 1, wherein the signal transmitter and the signal receiver are simultaneously set at forefeet or simultaneously set at heels.

12. A device for correcting a standing posture, comprising:
a signal transmitter, to transmit a signal;
a signal receiver, wirelessly connected to the signal transmitter, to receive the signal transmitted by the signal transmitter;
a processor, connected to the signal receiver, to determine whether strength of the signal received by the signal receiver is in a preset strength range and generate a prompting signal if the strength of the signal received by the signal receiver is not in the preset strength range; and
a prompter, connected to the processor, to prompt a user that the standing posture is not right after receiving the prompting signal.

13. A pair of shoes, comprising: the device for correcting the walking posture of claim 1.

14. A pair of insoles, comprising: the device for correcting the walking posture of claim 1.

15. A method for correcting a walking posture, adopting the device for correcting the walking posture of claim 1, comprising:
transmitting a signal using a signal transmitter, which is worn on a foot of a user, and receiving the signal transmitted by the signal transmitter using a signal receiver, which is worn on another foot of the user;
determining whether strength of the signal received by the signal receiver is in a preset strength range and generating a prompting signal if the strength of the signal received by the signal receiver is not in the preset strength range; and
prompting the user that the walking posture is not right according to the prompting signal.

16. The method according to claim 15, wherein before determining whether the strength of the signal received by the signal receiver is in the preset strength range, the method further comprises:
storing preset strength ranges corresponding to different walking modes, wherein the walking modes comprise: one or more of normal walking, fast walking, fast running and jogging; and
selecting one of the walking modes;
wherein determining whether the strength of the signal received by the signal receiver is in the preset strength range comprises:
obtaining a preset strength range corresponding to the currently-selected walking mode and determining whether the strength of the signal received by the signal receiver is in the preset strength range corresponding to the currently-selected walking mode.

17. The method according to claim 16, wherein before storing the preset strength ranges corresponding to the different walking modes, the method further comprises: setting the preset strength ranges, wherein the preset strength ranges are strength ranges of signals received by the signal receiver when walking postures of different walking modes of the user who respectively wears the signal transmitter and the signal receiver on his/her feet are right.

18. The method according to claim 15, wherein when determining that the strength of the signal received by the signal receiver exceeds the preset strength range, generating the prompting signal specifically comprises:
after the strength of the signal received by the signal receiver is determined to exceed the preset strength range, determining whether the walking posture of the user is an out-toe posture or an in-toe posture according to whether the strength of the signal received by the signal receiver is less than the minimum value of the preset strength range or larger than the maximum value of the preset strength range and generating a prompting signal comprising out-toe information or in-toe information.

19. The method according to claim 15, wherein when determining that the strength of the signal received by the signal receiver exceeds the preset strength range, generating the prompting signal specifically comprises:
after the strength of the signal received by the signal receiver is determined to exceed the preset strength range, determining a deviation level of the walking posture of the user according to a deviation value between the strength of the signal received by the signal receiver and the preset strength range and generating the prompting signal comprising the deviation level.

20. A method for correcting a standing posture, adopting the device for correcting the standing posture of claim 12, comprising:
transmitting a signal using a signal transmitter, which is worn on a foot of a user, and receiving the signal transmitted by the signal transmitter using a signal receiver, which is worn on another foot of the user;
determining whether strength of the signal received by the signal receiver equals to a preset strength range and generating a prompting signal if the strength of the signal received by the signal receiver does not equal to the preset strength range; and
prompting the user that the standing posture is not right according to the prompting signal.

21. A pair of shoes, comprising: the device for correcting the standing posture of claim 12.

22. A pair of insoles, comprising: the device for correcting the standing posture of claim 12.
